(19) United States
(12) Patent Application Publication
(10) Pub. No.: US 2026/0047587 A1
(43) Pub. Date: Feb. 19, 2026

(54) FEED COMPOSITION, METHOD OF PREPARING A FEED AND FEED

(71) Applicant: BERG + SCHMIDT GMBH & CO. KG, Hamburg (DE)

(72) Inventors: Andrew Philip Robinson, Hamburg (DE); Dirk Westphal, Hamburg (DE)

(21) Appl. No.: 19/102,168

(22) PCT Filed: Aug. 9, 2022
(86) PCT No.: PCT/EP2022/072333

§ 371 Date: Feb. 7, 2025

(51) Int. Cl. A23K 20/158; A23K 20/24; A23K 50/10; A61K 31/20; A61K 33/06

(52) U.S. Cl. CPC A23K 20/158; A23K 20/24; A23K 50/10; A61K 31/20; A61K 33/06

(57) ABSTRACT

The present invention relates to a feed composition for pre-paring a feed, comprising: a. a fatty acid mixture comprising: i. ≤50 wt. %, preferably 50-80 wt. %, further preferably 55-65 wt. %, more preferably 57-63 wt. %, even more preferably 58-63 wt. % of a C16:0 fatty acid, based on the total amount of the fatty acid mixture. ii. ≤40 wt. %, preferably 15-40 wt. %, further preferably 20-37 wt. %, more preferably 25-35 wt. %, even more preferably 26-30 wt. % of a C18:1 fatty acid or mixtures thereof, based on the total amount of the fatty acid mix-ture, b. 0.001-8 wt. % of an alkali metal, an alkali metal or mixtures thereof, based on the total proportion of the feed composition; a method for preparing a feed, and a feed.

The invention relates to a feed composition for preparing a feed, a feed prepared from such a feed composition, a method for preparing a feed, and a feed prepared from such a method.

Most dairy cows have very high levels of milk production, which results in the cows having a high energy demand. At the beginning of lactation, most cows lose considerable weight because the energy intake is lower than the nutrient requirements for milk production. As a result, the cow mobilizes the body's nutrient reserves, especially body fat, to meet the energy demand. Therefore, giving dairy cows the right feed is of immense importance.

At the moment, there basically exist different animal feeds, particularly rumen bypass fats for cows, which basically can be classified in Ca-soap, hydrogenated fat, and fractionated fat products.

Hydrogenated fat products essentially contain a higher content of the fatty acid C18:0 than Ca-soap or fractionated fat products. This leads to less fat digestibility in comparison to fats or products with higher content of the fatty acid C16:0.

Ca-soap products consist of fat esterified with alkaline earth ions to make it rumen stable. However, products known in the prior art (such as, Mega-Max (Volac Wilmar)), have a pungent smell and taste. This can reduce the animal's feed intake. The fat content of Ca-soap products is low (about 84%), the content of alkaline earth ions is about 11% and they contain approx. 5% moisture and impurities. In addition, Ca-soap products show a comparatively low stability at a pH≤5.6. This is disadvantageous, since, for example, during feeding diets with high starch content to ruminants the ruminal pH-level drops and as a consequence of low pH-level the Ca-soap breaks. In addition, Corn silage, which is the major feed component in most farming systems, has a pH 4. Ca-soaps can also be broken up in the diet before they are eaten by the animal.

Fractionated fats have a high fat content with high amounts on the fatty acids C16:0 and C18:1, but the slip melting point is not as high as needed in some regions for livestock feed. Particularly, the C18:1 fatty acid oleic acid is very “soft” with a low melting point. This softness makes the fractioned fatbased products unsuitable for handling, transport and storage.

Therefore, there is a need for a feed composition and an animal feed that overcomes the disadvantages known in the prior art.

The invention is concerned with the problem of providing a feed composition, a method for preparing a feed, as well as a feed, which overcome the above-mentioned disadvantages of the prior art and which, in particular, makes it possible to provide a feed (composition), particularly a rumen bypass fat for cows, having improved properties, especially a high fat content, an optimal fatty acid composition and good stabilities at high temperatures.

This problem is solved by the features of the independent claims. Advantageous embodiments are defined in the dependent claims.

In particular, the invention is directed to a feed composition for preparing a feed, comprising:
a. a fatty acid mixture, which comprises:
   i. 50-80 wt. %, preferably 55-65 wt. %, more preferably 57-63 wt. %, even more preferably 58-63 wt. % of a C16:0 fatty acid, based on the total amount of the fatty acid mixture,
   ii. 15-40 wt. %, preferably 20-37 wt. %, more preferably 25-35 wt. %, even more preferably 26-30 wt. % of a C18:1 fatty acid or mixtures thereof, based on the total amount of the fatty acid mixture,
b. 0.001-5 wt. % of an alkali metal, an alkaline earth metal or mixtures thereof, based on the total amount of the feed composition.

The invention is defined by the claims. Embodiments contained in the description, which are not the subject of the claims, merely serve to explain individual aspects of the invention.

First, several terms are explained in the context of the invention.

In the context of the present invention the term “feed composition” is defined as a composition comprising or consisting a certain fatty acid mixture (or fatty acid profile) and a certain amount of alkali ions and/or alkaline earth ions, preferably Ca-ions, which are used as starting materials for preparation a feed, preferably an animal, further preferably ruminants. I.e., from the feed composition the corresponding feed is obtained.

In the context of the present invention the term “feed” includes also diet, nutrient and supplemental feed products, such as fat supplements, energy supplements, rumen stable supplements etc. Preferably, the feed according to the present invention is a supplemental feed, further preferably a supplement fat feed, supplement energy feed, and/or supplement rumen stable feed. In the context of the present invention a “supplemental feed” is defined as a secondary feed source used to provide additional attributes, both required and optional, that may be missing from a primary feed source. Supplemental feeds cannot be used as a main feed source.

In the context of the present invention the term C16:0 fatty acid relates to a 16-carbon saturated fatty acid (hexadecanoic acid), e.g. palmitic acid.

In the context of the present invention the term C18:1 fatty acid relates to a 18-carbon monounsaturated fatty acid (octadecenoic acid), such as oleic acid, vaccenic acid, elaidic acid, petroselinic acid, etc. Preferably, the C18:1 fatty acid is oleic acid (z-9-octadecenoic acid).

Preferably, the alkali metal and/or the alkaline earth metal content in the feed composition is 0,01-3 wt. %, more preferably 0,1-2 wt. %, based on the total amount of the feed composition.

In the context of the present invention it is preferred that the alkali metal and/or alkaline earth metal is selected from sodium, potassium, calcium, magnesium, and mixtures thereof. In preferred embodiment, the alkaline earth metal is calcium.

It is preferred that the fatty acid mixture of the feed composition further comprises one or more fatty acids or fatty acid mixtures selected from:
iii. a C18:0 fatty acid,
iv. a fatty acid or a fatty acid mixture selected from C8-C14 fatty acids,
V. a fatty acid or a fatty acid mixture selected from C18:2, C18:3, C20:0, and C20:1 fatty acids.

In the context of the present invention the term C18:0 fatty acid relates to a 18-carbon saturated fatty acid (octadecanoic acid), e.g. stearic acid.

In the context of the present invention the term C8-C14 fatty acids relates to saturated and unsaturated fatty acid having 8 to 14 carbon atoms. Preferably, the C8-C14 fatty acids are selected from C8, C10, C12, and C14 fatty acids. Preferred saturated fatty acids are caprylic acid, capric acid, lauric acid, and myristic acid. Preferred unsaturated fatty acids are monounsaturated fatty acids, more preferred myristoleic acid (C14:1). In a preferred embodiment the C8-C14 fatty acid is lauric acid and/or myristic acid.

In the context of the present invention the term C18:2 fatty acid relates to a 18-carbon unsaturated fatty acid (octadecadienoic acid) containing two double bonds in its back bone. Examples for C18:2 fatty acids are linoleic acid, and linolelaidic acid. Preferably, the C18:2 fatty acid is linoleic acid.

In the context of the present invention the term C18:3 fatty acid relates to a 18-carbon unsaturated fatty acid (octadecatrienoic acid) containing three double bonds in its back bone. Examples for C18:3 fatty acids are alpha-linolenic acid, gamma-linolenic acid, calendic acid, pucinic acid, alpha-eleostearic acid, beta-eleostearic acid. Preferably, the C18:3 fatty acid is alpha-linolenic acid.

In the context of the present invention the term C20:0 fatty acid relates to a 20-carbon saturated fatty acid (e.g. icosanoic acid). Preferably, the C20:0 fatty acid is arachidic acid.

In the context of the present invention the term C20:1 fatty acid relates to a 20-carbon unsaturated fatty acid (eicosenoic acid) containing one double bond in its back bone. Examples for C20:1 fatty acids are gadoleic acid, and gondocic acid. Preferably, the C20:1 fatty is gadoleic acid, gondocic acid and mixtures thereof.

Furthermore, it is also conceivable that other fatty acids commonly used in animal feed can be used in the inventive fatty acid mixture. Such fatty acids can be, for example: C4:0 (butyric acid), C6:0 (caproic acid), C22:1 (erucic acid), (20:4 arachidonic acid), 20:5 (eicosapentaenoic acid), C22:6 (docosahexaenoic acid), and mixtures thereof.

In preferred embodiments, the fatty acid mixture does not contain other fatty acids than the ones defined above in i. to v. In this case, the fatty acid mixture preferably consists of i. a C16:0 fatty acid, ii. a C18:1 fatty acid or mixtures thereof, optionally iii. a C18:0 fatty acid, optionally iv. a fatty acid or fatty acid mixture selected from C8-C14 fatty acids, and optionally v. a fatty acid or fatty acid mixture selected from C18:2, C18:3, C20:0, and C20:1 fatty acids.

It is preferred that one or more fatty acids are comprised in the fatty acid mixture in each of the following amounts, based on the total amount of the fatty acid mixture:
iii. ≤4 wt. %, preferably ≤3 wt. %, more preferably 0.1-3 wt. %, still more preferably 1-3 wt. % of a C18:0 fatty acid,
iv. ≤2 wt. %, preferably 0.1-2 wt. %, more preferably 1-2 wt. %, still more preferably 1.5-2 wt. % of a fatty acid or fatty acid mixture selected from C8-C14 fatty acids,
V. ≤10 wt. %, preferably 0.1-10 wt. %, more preferably 3-10 wt. %, still more preferably 5-10 wt. % of a fatty acid or fatty acid mixture selected from C18:2, C18:3, C20:0, C20:1 fatty acids.

Preferably, the amount of C18:2 fatty acid is ≤10 wt. %, more preferably is 5-9 wt. %, still more preferably is approx. 7 wt.-%, based on the total amount of the fatty acid mixture.

Preferably, the amount of each of C18:3, C20:0 and C20:1 fatty acid is <0.5 wt.-%, based on the total amount of the fatty acid mixture.

Further, the invention is directed to a method for preparing a feed composition, comprising:
a. providing a fatty acid mixture, which comprises:
   i. 250 wt. %, preferably 50-80 wt. %, further preferably 55-65 wt. %, more preferably 57-63 wt. %, even more preferably 58-63 wt. % of a C16:0 fatty acid, based on the total amount of the fatty acid mixture,
   ii. ≤40 wt. %, preferably 15-40 wt. %, further preferably 20-37 wt. %, more preferably 25-35 wt. %, even more preferably 26-30 wt. % of a C18:1 fatty acid or mixtures thereof, based on the total amount of the fatty acid mixture,
b. adding 0.001-8 wt. % of an alkali metal, an alkaline earth metal or a mixture thereof, based on the total amount of the feed composition, to the fatty acid mixture provided in step a.

According to one preferred embodiment the alkali metal, the alkaline earth metal or the mixture thereof is added to the fatty acid mixture provided in step a. It is preferred that the addition is carried out with stirring.

Preferably the alkali metal, the alkaline earth metal or the mixture thereof is added in form of an alkali metal soap, an alkaline earth metal soap or a mixture thereof. More preferably Ca-soap is added. According to another preferred embodiment the alkali metal, the alkaline earth metal or the mixture thereof is added in form of an oxide, more preferably Ca-oxide is added. In the process with the oxide, water can be added to support the reaction.

Preferably the fatty acid mixture of step a. is heated to temperature in the range of 50-140° C., further preferably 60-130° C., further preferably 65-120° C. for the addition of the alkali metal, the alkaline earth metal or the mixture thereof. When alkali metal soap, alkaline earth metal soap or a mixture thereof is added, the fatty acid mixture is preferably heated to a temperature in the range of 60-130° C., further preferably 70-120° C. When an oxide is added, the fatty acid mixture of step a. is preferably heated to temperature in the range of 60-80° C., further preferably 65-70° C.

Furthermore, the invention is directed to a feed, prepared from an inventive feed composition, preferably a feed composition according to any one of claims 1 to 6.

Furthermore, the invention is directed to a feed, prepared according to the inventive method, preferably prepared by the method according to any one of claims 7 to 9.

Preferably, the feed has a fat content higher than 85 wt. %, further preferably higher than 90 wt. %, further preferably higher than 95 wt. %, still further preferably higher than 97 wt. %, based on the total amount of the feed composition. The fat content is measured after fat extraction and determined by Werner-Schmid method.

In preferred embodiments, the feed has a slip melting point in a range of 60-90° C., preferably of 65-80° C., further preferably of 70-76° C., still further preferably of about 71° C., measured according to AOCS Official method Cc 3-25, Slip melting point AOCS Standard Open Tube Melting Point, 2009.

Preferably, a moisture content of the feed is less than 10 wt. %, further preferably less than 5 wt. %, further preferably less than 4 wt. %, further preferably is 3 wt. %, based on the total amount of the feed composition. To determine the moisture content of the feed, the sample is weighed, dried at 105° C. for 4 hours and then weighed again.

In the context of the invention the feed is preferably an animal feed, further preferably a feed for ruminants, further preferably a feed for fattening cattle and/or lactating dairy COWS.

In preferred embodiments, the feed is in the form of beads, pellets, a powder or flakes, preferably is in bead form.

The features described for the feed composition according to the invention, in particular the ingredients and amounts used, also apply, where appropriate, to the method and feed according to the invention.

Further, the invention is directed to the use of the inventive feed as a supplemental feed, preferably a supplement fat feed, supplement energy feed, and/or supplement rumen stable feed.

The invention has surprisingly recognized that the inventive feed (composition) has improved properties like high fat content, optimal fatty acid composition and good stability at high temperatures and also pH≤5.6, compared to other products available on the market, especially rumen bypass fats. This is because the inventive feed (composition) is not a Ca-soap or a fractionated fat product, but rather represents a modified fractionated fat.

It has been surprisingly found that the alkali ions and alkaline earth ions used do not serve for making the fat rumen stable. It is already rumen stable, since it is based on fractionated fat. The alkali ions and/or alkaline earth ions, preferably the Ca-ions, used in the present invention give the feed the property to be more stable under higher temperatures in comparison to typical fractionated fat.

Nevertheless, the amount of Ca-ions used in the present invention is low enough to have no adverse effects on the feed. Ca-soaps, for example, have a higher Ca content, and therefore have a pungent smell and taste, which can reduce the animal's feed intake. This is not the case for the inventive feed.

In addition, the optimal fatty acid mixture (or profile) of the invention has a major effect on nutrient digestibility, energy partitioning, and production responses of cattle, and particularly dairy cows. Feeds with higher C16:0 fatty acid content increase milk and milk fat production while higher C18:1 fatty acid amounts increase body condition. Therefore, the inventive feed with high levels of C16:0 and C18:1 fatty acid increase milk fat and at the same time increases fat digestibility and fertility. This means that the feed according to the invention is not only more palatable, but also has a good fatty acid composition for bioavailability for cattle and especially lactating dairy cows.

In the following, advantageous embodiments will be explained by way of example.

### EXAMPLES

a. Feed Compositions

In the following Table 1 feed compositions and their resulted feed properties are listed.

Example 1 (Ex.1) represents a preferred embodiment of the inventive feed composition. Comparative Example 1 (Comp.Ex.1) shows the composition of the prior art Ca-soap feed product Mega-Max (Volac Wilmar).

TABLE 1
Inventive feed composition (Ex. 1) and the composition of a prior
art Ca-soap feed (Comp. Ex. 1) and their resulted feed properties

| | Ex. 1 | Comp. Ex. 1 |
|---|---|---|
| Ca content, wt. % | 1 | 9 |
| C8:0 to C14:0 | 1.9 | |
| C16:0 | 62.8 | 58 |
| C18:0 | 2.0 | 5 |
| C18:1 | 27.8 | 28 |
| C18:2 and more | 5.5 | 6 |
| Other fatty acids | — | 3 |
| Total fatty acids, wt. % | 100 | 100 |
| Slip melting point, °C. | 71 | <100 |
| Unfavorable odor | no | yes |

b. Comparison of Different Feed Types

In the following Table 2 an inventive feed and its properties is compared to different feeds, particularly rumen bypass fats, currently available on the market (Comparative Examples 1-3, Comp. Ex. 1-3).

Example 2 (Ex. 2) relates to a feed according to the invention. Comparative Example 2 (Comp. Ex. 2) is a hydrogenated fat product. Comparative Example 3 (Comp. Ex. 3) is the Ca-soap product Mega-Max (Volac Wilmar), which has the feed composition as listed in the above Comparative Example 1 (Comp. Ex.1). Comparative Example 4 relates to the fractionated fat product BergaFat F-100 (Berg+Schmidt).

TABLE 2
Inventive feed (Ex. 2) and its properties compared to current
rumen bypass fats available on the market (Comp. Ex. 2-4)

| | | Comp. | Comp. | Comp. |
|---|---|---|---|---|
| | Ex. 2 | Ex. 2 | Ex. 3 | Ex. 4 |
| Form | beads | beads | granules/ | beads |
| | | | powder | |
| Dust | no | no | up to 40% | no |
| Unfavorable odor | no | no | yes | no |
| Palatability | yes | yes | no | yes |
| (no reduction of | | | | |
| feed intake) | | | | |
| Energy content, NEL | ~26 | ~26 | ~20.4 | ~26 |
| MJ/kg | | | | |
| Ca content, % | <2 | 0 | ~9 | 0 |
| Fat content, % | >98 | >99 | ~84 | >98 |
| Fatty acid mixture: | | | | |
| C16:0, wt. % | >58 | >45 | ~48 | >80 |
| C18:0, wt. % | <4 | >45 | ~5 | ~5 |
| C18:1, wt. % | >25 | <1 | ~36 | ~6.7 |
| No melting up to 50° C. | yes | yes | yes | yes |
| No melting up to 60° C. | yes | no | yes | no |
| No melting up to 70° C. | yes | no | yes | no |
| Rumen stability, % | >95 | >95 | ~80 (not | >95 |
| | | | stable at | |
| | | | low pH) | |
| Digestibility, % | >85 | ~70 | ~85 | ~84 |

### Measurement Methods

a. Fat Content

The fat content was measured after fat extraction and determined by Werner-Schmid method.

In this method, the sample (dryed, 100° C.) is dissolved in concentrated hydrochloric acid and then extracted with diethyl ether and petroleum ether (1:1). Afterwards, the solvents are removed by distillation. The dried residue is weighted. The fat content is calculated from the difference of the weights of the original flask and the flask plus extracted fat.

b. Slip Meting Point

The slip melting point of the feed was measured according to AOCS Official method Cc 3-25, Slip melting point AOCS Standard Open Tube Melting Point, 2009.

According to this method, the sample is melted and filtered to guarantee no moisture or impurities in it. Three capillaries are dipped in the sample until a rise of 10 mm high in the tubes, then the samples were incubated by 4-10° C. for 16 hours. Afterwards, the samples are put in a water bath with a temperature 10° C. below the suspected sip point. The temperature is rised in steps of 1° C. The observed temperature, where each sample column is rising, is taken and the average of the three samples is determined as the slip point.

1. Feed composition for preparing a feed, comprising:
a. a fatty acid mixture, which comprises:
i. 50-80 wt. %, preferably 55-65 wt. %, more preferably 57-63 wt. %, even more preferably 58-63 wt. % of a C16:0 fatty acid, based on the total amount of the fatty acid mixture,
ii. 15-40 wt. %, preferably 20-37 wt. %, more preferably 25-35 wt. %, even more preferably 26-30 wt. % of a C18:1 fatty acid or mixtures thereof, based on the total amount of the fatty acid mixture,
b. 0.001-5 wt. % of an alkali metal, an alkaline earth metal or mixtures thereof, based on the total amount of the feed composition.

2. Feed composition according to claim 1, characterized in that the alkali metal and/or alkaline earth metal content in the feed composition is 0.001-5 wt. %, further preferably 0,01-3 wt. %, more preferably 0,1-2 wt. %, based on the total amount of the feed composition.

3. Feed composition according to claim 1, characterized in that the alkali metal and/or alkaline earth metal is selected from sodium, potassium, calcium, magnesium, and mixtures thereof.

4. Feed composition according to claim 3, characterized in that the alkaline earth metal is calcium.

5. Feed composition according to claim 1, characterized in that the fatty acid mixture further comprises one or more fatty acids or fatty acid mixtures selected from:
iii. a C18:0 fatty acid,
iv. a fatty acid or a fatty acids mixture selected from C8-C14 fatty acids,
V. a fatty acid or fatty acid mixture selected from C18:2, C18:3, C20:0, and C20:1 fatty acids.

6. Feed composition according to claim 5, characterized in that one or more fatty acids is/are comprised in the fatty acid mixture in each of the following proportions, based on the total proportion of the fatty acid mixture:
iii. ≤4 wt. %, preferably ≤3 wt. %, more preferably 0.1-3 wt. %, still more preferably 1-3 wt. % of a C18:0 fatty acid,
iv. ≤2 wt. %, preferably 0.1-2 wt. %, more preferably 1-2 wt. %, still more preferably 1.5-2 wt. % of a fatty acid or fatty acid mixture selected from C8-C14 fatty acids,
V. ≤10 wt. %, preferably 0.1-10 wt. %, more preferably 3-10 wt. %, still more preferably 5-10 wt. % of a fatty acid or a fatty acid mixture selected from C18:2, C18:3, C20:0, and C20:1 fatty acids.

7. Method for preparing a feed, comprising:
a. providing a fatty acid mixture, which comprises:
i. 50-80 wt. %, preferably 55-65 wt. %, more preferably 57-63 wt. %, even more preferably 58-63 wt. % of a C16:0 fatty acid, based on the total amount of the fatty acid mixture,
ii. 15-40 wt. %, preferably 20-37 wt. %, more preferably 25-35 wt. %, even more preferably 26-30 wt. % of a C18:1 fatty acid or mixtures thereof, based on the total amount of the fatty acid mixture,
b. adding 0.001-5 wt. % of an alkali metal, an alkaline earth metal or mixtures thereof, based on the total amount of the feed composition, to the fatty acid mixture provided in step a.

8. (canceled)

9. Method for preparing a feed according to claim 7, characterized in that the alkali metal, the alkaline earth metal or mixture thereof is added in form of an alkali metal soap, an alkaline earth metal soap or a mixture thereof, preferably Ca-soap, or is added in form of an oxide, preferably Ca-oxide.

10. Feed, prepared from a feed composition according to claim 1.

11. Feed according to claim 10, characterized in that the feed has a fat content higher than 85 wt. %, preferably higher than 90 wt. %, further preferably higher than 95 wt. %, still further preferably higher than 97 wt. %, based on the total amount of the feed composition.

12. Feed according to claim 10, characterized in that the feed has a slip melting point in a range of 60-90° C., preferably of 65-80° C., further preferably of 70-76° C., still further preferably of about 71° C.

13. Feed according to claim 10, characterized in that it is an animal feed, preferably a feed for ruminants, further preferably a feed for fattening cattle and/or lactating dairy cows.

14. Feed according to claim 10, in the form of beads, pellets, a powder or flakes, preferably in bead form.

15. Use of the feed according to claim 10 as a supplemental, preferably a fat supplement fat, energy supplement, and/or rumen stable supplement.
